(19) United States
(12) Patent Application Publication
(10) Pub. No.: US 2022/0175710 A1
(43) Pub. Date: Jun. 9, 2022

(54) COMBINATION THERAPY

(71) Applicant: Akershus Universitetssykehus HF, Lørenskog (NO)

(72) Inventors: Tormod FLADBY, Lørenskog (NO); Marianne WETTERGREEN, Lørenskog (NO); Silje TORSETNES, Lørenskog (NO); Berglind GISLADOTTIR, Lørenskog (NO); Kaja NORDENGEN, Lørenskog (NO)

(73) Assignee: Akershus Universitetssykehus HF, Lørenskog (NO)

(21) Appl. No.: 17/604,371

(22) PCT Filed: Apr. 20, 2020
(86) PCT No.: PCT/EP2020/061018

§ 371 Date: Oct. 15, 2021

(30) Foreign Application Priority Data
Apr. 18, 2019 (EP) 19170285.1

(51) Int. Cl. A61K 31/202; A61K 31/4402; A61K 31/196; A61K 31/192; A61K 31/55; A61K 31/17; A61K 31/42; A61K 31/473; A61K 31/439; A61K 31/5377; A61K 31/4425; A61K 31/501

(52) U.S. Cl. CPC A61K 31/202; A61K 31/4402; A61K 31/196; A61K 31/192; A61K 31/55; A61K 31/501; A61K 31/42; A61K 31/473; A61K 31/439; A61K 31/5377; A61K 31/4425; A61K 31/17

(57) ABSTRACT

A combination of an FFAR4 agonist and an α7 nAChR agonist or positive modulator. The combination is useful for the treatment of neurodegenerative diseases.

### FIELD OF THE INVENTION

The invention relates to a combined preparation or composition comprising an FFAR4 agonist and an α7 nAChR agonist or positive modulator. The invention also relates to the use of an FFAR4 agonist and an α7 nAChR agonist or positive modulator, in combination, for the treatment of neurodegenerative diseases.

### BACKGROUND OF THE INVENTION

Alzheimer's disease (AD) is known to be associated with amyloid beta (Aβ), which is a 38-43 amino acid (aa) peptide (isoforms from 38-43 aa) derived from amyloid precursor protein and is deposited in amyloid plaques. In particular, the 42 and 43 aa forms polymerizes to oligomers and fibrils, which are neurotoxic, although polymerization and toxicity is retained even in the partly-catabolized shorter forms. We have earlier demonstrated patterns of Aβ catabolism due to Endoplasmic Reticulum-derived enzymes (Rogeberg et al. 2014). Synapse loss is an early feature of Alzheimer's disease and is currently thought to be linked to Aβ dysmetabolism. Reduced cholinergic function is also an early feature of Alzheimer's disease, which is insufficiently mitigated by symptomatic cholinergic treatments (e.g. Donepezil, Galantamine, Exelon). Progression towards AD is also characterised by increased microglial activation and inflammation (Nordengen et al. 2019).

In vivo, the central nervous system (CNS) innate immune cells, including microglia (bone marrow stem-cell derived cells, seeded to the CNS during gestation and upheld as cell population by local proliferation), uphold synaptic homeostasis. This includes phagocytosis and degradation of activity-induced Aβ production, in an intricate network with pre- and postsynaptic cells/compartments, as well as astroglia. The initial sequence of events is not fully understood, although it is currently thought that microglia properties change in incipient AD, and acquire an inflammatory phenotype as the patient progresses towards AD-induced dementia. Microglia are myelogenous brain-resident innate immune cells and are main and early responders in the CNS immune defence. They are also thought to play a role in upholding of synaptic homeostasis.

During ageing, Aβ half-life increases, which is thought to contribute to age-related increase in AD incidence. Communication between the peripheral immune system and microglia leads to an increase in circulation of peripheral blood innate immune cells (monocytes) to the CNS in pathological situations. Peripheral myeloid cells, such as monocytes and macrophages, are regulated in parallel to the microglia histiocytes in many respects and share phagocytic properties. In addition, these cells may circulate to and infiltrate the CNS and are thought possibly to play a role in AD pathogenesis such as cerebral amyloidosis. The peripheral Aβ compartment (the compartment outside the CNS) functions as an Aβ sink for CNS. In general, 50% of Aβ catabolism is outside the CNS. Co-regulation of gene-expression profiles across innate immune cell types of monocytic lineage (microglia, monocytes and macrophages) have been described in established AD. Murine studies have demonstrated phagocytosis of fibrillar Aβ within bone marrow-derived macrophages; cerebral Aβ clearance by peripheral monocyte-derived macrophages (Koronyo et al. 2015); and have shown that impaired microglial phagocytosis coincides with Aβ plaque deposition (Koronyo et al. 2015; Zuroff et al. 2017; Krabbe et al. 2013).

Polyunsaturated fatty acids (PUFAs), including omega-3 fatty acids, are important constituents of the phospholipids of all cell membranes. Modification of innate immune activity has already been seen using Docosahexaenoic acid (DHA; IUPAC name (4Z, 7Z, 10Z, 13Z, 16Z, 19Z)-4, 7, 10, 13, 16, 19-docosahexaenoic acid))—rich supplements, and this type of intervention has been shown to ameliorate AD-associated PBMC (peripheral blood mononuclear cell) and microglia profiles, and to be associated with improvements in cognition (Wang et al. 2015; Antonietta et al. 2012). Wang et al. demonstrated that Abeta-40, a common form of Aβ, decreases the production of specialized proresolving mediators (SPMs), which play a key role in the resolution of inflammation, by peripheral blood mononuclear cells (PBMCs). Wang et al. further demonstrated that treatment of AD patients with an oil rich in DHA prevented the reduction in production of SPMs from PBMCs, and that this was associated with improvements in cognition. Antonietta et al. demonstrated that DHA inhibits LPS-induced production of pro-inflammatory cytokines (such as TNF-α, IL-6 and IL-1β) and nitric oxide by microglia in a dose-dependent manner in vitro. Peripheral blood monocytes (PBM) are also bone marrow stem-cell derived, but with a short half-life (1-7 days) in the blood and replenished continuously from the bone-marrow.

Other studies have also shown that omega-3 fatty acids such as DHA have protective, anti-inflammatory effects on adipocytes and macrophages (Alvarez-Curto et al. 2016; Im 2015). Omega-3 fatty acids, such as DHA, activate FFAR4 receptors, which inhibit effects of inflammatory stimuli like LPS and downregulate the NF-kB system (Alvarez-Curto et al. 2016), which leads to modulation and mitigation of inflammatory responses.

WO 2011/006144 discloses methods of treating and preventing neurological disorders using DHA.

DHA crosses the BBB (blood-brain barrier), and resulting cerebro-spinal fluid (CSF) concentrations are associated with reduced CSF total tau levels, indicating that they reduce neurodegeneration, ameliorate Abeta-induced neuronal damage, and increase microglia Aβ phagocytosis (Antonietta et al. 2012; Freund et al. 2014; Tan et al. 2016).

In another field, WO 2018/150276 discloses the use of cotinine and krill oil for the treatment of chronic stress and depression, particularly PTSD.

As mentioned above, cholinergic treatments only insufficiently mitigates cognitive symptoms associated with Alzheimer's disease, and have not been shown to mitigate disease progression. Thus, there is a need for improved treatments for neurodegenerative diseases such as Alzheimer's disease.

In a different technical field, Lappe et al. report on the effect of genistein, polyunsaturated fatty acids and vitamins D3 and K1 on bone mineral density in postmenopausal women.

### SUMMARY OF INVENTION

The present invention arises because it has now, surprisingly, been shown that DHA treatment of cells in an innate immune model system increases Aβ phagocytosis as well as degradation. The results (shown in the Examples below) indicate that increased Aβ phagocytosis and degradation may be mediated in part by increased activity of Endoplasmic Reticulum (ER)-related enzymes(1), consistent with positive effects of DHA on ER stress(2). It is now understood that the effects of DHA seen on Aβ phagocytosis and degradation are mediated via FFAR4 receptors, and that increased Aβ phagocytosis is mediated by increased CHRNA7-expression at the plasma membrane (3).

The increased microglial activation and inflammation seen in Alzheimer's disease will be accompanied by increased NF-kB-activity, and by reduced and insufficient CHRNA7 expression at the membrane and reduced cholinergic responsivity.

Neuroinflammation is regulated in part through the neuroimmune axis, where stimulation of α7-nicotinic receptors (α7 nicotinic acetylcholine receptors; α7 nAChR) on innate immune cells is an important component (4)(5). Innate immune α7-cholinergic activation ameliorates inflammatory activation. CHRNA7 is the gene for the classic α7 nAChR receptor, expressed inter alia on neurons and innate immune cells.

CHRFAM7A is a nearby uniquely human gene partially duplicated from CHRNA7. CHRFAM7A transcription or expression is known to hinder CHRNA7 expression or α7 nAChR function, most likely promoting CNS inflammatory activation and putatively hindering synaptic nicotinic transmission (6-8). The α7 nAChR is a pentamer, with a major homomeric form (CHRNA7), but can be pseudo-heteromeric in that α7 monomers from CHRFAM7A may intersperse the otherwise homomeric pentamer. The CHRFAM7A gene is present in a variable number of copies, contains a high number of polymorphisms that are associated with several neuropsychiatric diseases and likely reduces α7 nAChR expression and function (9-10).

Therapeutic modulation and activation of the α7 nicotinic system is used for treatment of e.g. Alzheimer's disease, Schizophrenia, Parkinson's disease, but further treatment efficacy is sought for all diseases (9). CNS inflammation also accompanies and may cause disease progression or treatment resistance but is not a part of the current treatment repertoire (11-13).

It is also proposed that a cholinergic insufficiency may be self-reinforcing, in that lack of α7 nicotinic stimulation will lead to stronger inflammatory activation and even further reduced CHRNA7 expression (King et al., 2017). In addition, it has been found that Aβ fibrils bind α7 nAChR and are subsequently phagocytosed, such that a lack of plasma membrane α7 nicotinic receptors will also reduce fibrillar Aβ-phagocytosis and fibrillar Aβ-α7-mediated anti-inflammatory signaling (Rothbard et al. 2018).

The present invention is based on the understanding that FFAR4 agonists, such as omega-3 fatty acids (for example, DHA), constitutively mitigate NF-kB activation, inflammatory activation. We hypothesized, tested and confirmed that this also increases CHRNA7 expression (FIG. 4), allowing both physiologic and pharmacologic cholinergic stimulation to have effect and thus impeding AD progression. In particular, FFAR4 activation inhibits NF-kB, which leads to an increase in CHRNA7 expression, as well as a reduced inflammatory response. The increased expression of CHRNA7 would result in increased Aβ phagocytosis.

However, intracellular accumulation of Aβ contributes to AD pathogenesis, and increased Aβ phagocytosis cannot be expected to ameliorate AD in the absence of associated increased degradation. The present invention is based on the realization that FFAR4 and α7 nicotinic stimulation can be expected to act in synergy, by both increasing Aβ phagocytosis and degradation (FIG. 3) by increasing the function of physiologic reaction pathways.

Thus, in a first aspect the present invention provides a combined preparation comprising an FFAR4 agonist and an α7 nAChR agonist or positive modulator.

In a second aspect, the present invention provides a composition comprising an FFAR4 agonist and an α7 nAChR agonist or positive modulator.

Conveniently, the α7 nAChR agonist or positive modulator is a positive allosteric modulator.

Preferably, the positive allosteric modulator is Galantamine, NS-1738, PNU-120596 or TQS, or a pharmaceutically acceptable salt thereof.

Alternatively, the α7 nAChR agonist or modulator is an agonist.

Conveniently, the agonist is PNU-282987, SEN 12333, TC 5619 S24795 or A-582941,or a pharmaceutically acceptable salt thereof.

Preferably, the α7 nAChR agonist or positive modulator is a Type I PAM, more preferably is selected from the group consisting of Genistein, NS-1738, AVL-3288 and Galantamine.

Alternatively, the α7 nAChR agonist or positive modulator is a Type II PAM, preferably selected from the group consisting of PNU-120596 and PAM-2.

Preferably, the combined preparation or composition comprises more than one α7 nAChR positive modulator.

Advantageously, the more than one α7 nAChR positive modulator comprises Galantamine, NS-1738, PNU-120596 and TQS.

Conveniently, the FFAR4 agonist is a PUFA, Compound A, NCG 21, GW9508 or TUG-891, or a pharmaceutically acceptable salt thereof.

Advantageously, the PUFA is a long chain PUFA (C18 to 22).

Preferably the PU FA is an omega-3 fatty acid.

More preferably, the PUFA is DHA.

Advantageously, the combined preparation or composition comprises DHA, Galantamine, NS-1738, PNU-120596 and TQS.

Conveniently, the combined preparation or composition is a pharmaceutical composition and comprises a pharmaceutically-acceptable carrier, diluent or excipient.

In a third aspect of the present invention, there is provided a combined preparation or composition comprising an FFAR4 agonist and an α7 nAChR agonist or positive modulator, for use in a method of treating a neurodegenerative disease, wherein the combined preparation is in accordance with the first aspect of the invention and the composition is in accordance with the second aspect of the invention.

In a fourth aspect of the present invention, there is provided an FFAR4 agonist for use in a method of treating a neurodegenerative disease, wherein the method comprises simultaneous or sequential administration of the FFAR4 agonist with an α7 nAChR agonist or positive modulator.

In a fifth aspect of the present invention, there is provided an α7 nAChR agonist positive modulator for use in a method of treating a neurodegenerative disease, wherein the method comprises simultaneous or sequential administration of the α7 nAChR agonist or positive modulator with an FFAR4 agonist.

Conveniently, the FFAR4 agonist is a PUFA, Compound A, NCG 21, GW9508 or TUG-891, or a pharmaceutically acceptable salt thereof.

Advantageously, the PUFA is a long chain PUFA (C18 to 22).

Preferably, the PUFA is an omega-3 fatty acid.

More preferably, the PUFA is DHA.

Advantageously, the α7 nAChR agonist or positive modulator is a positive allosteric modulator.

Conveniently, the positive allosteric modulator comprises at least one of Galantamine, NS-1738, PNU-120596 and TQS, or a pharmaceutically acceptable salt thereof.

Preferably, the positive allosteric modulator comprises Galantamine, NS-1738, PNU-120596 and TQS.

Advantageously, the α7 nAChR agonist or positive modulator is an α7 nAChR agonist.

Conveniently, the α7 nAChR agonist is PNU-282987, SEN 12333, TC 5619, S24795 or A-582941, or a pharmaceutically acceptable salt thereof.

Preferably, the FFAR4 agonist is DHA and the α7 nAChR agonist or positive modulator comprises Galantamine, NS-1738, PNU-120596 and TQS.

Advantageously, the neurodegenerative disease is Alzheimer's disease.

According to a sixth aspect of the present invention, there is provided a kit comprising a first product comprising an FFAR4 agonist and a second product comprising an α7 nAChR agonist or positive modulator.

In a seventh aspect, the present invention provides a method of treating a neurodegenerative disease, comprising administering to a patient in need thereof a combined preparation as described in the first aspect of the invention or a composition as described in the second or third aspect; or an FFAR4 agonist as described in the fourth aspect and an α7 nAChR agonist or positive modulator as described in the fifth aspect above.

The term “FFAR4”, as used herein, refers to a free fatty acid receptor which is a member of the ‘rhodopsin-like’ G-protein couple receptor (GPCR) family, and which is activated selectively by long chain fatty acids. FFAR4 was previously known as GPR120. Further details thereof may be found in Free Fatty Acid Receptors, Springer, 2018, pp 33-56, which is incorporated herein by reference.

The term “α7 nAChR”, as used herein, refers to the nicotinic acetylcholine receptor made up of five identical α7subunits.

The term “agonist”, as used herein, refers to a substance which binds to and directly activates a receptor. It includes both full agonists and partial agonists (i.e. agonists which have only partial efficacy compared to a full agonist).

The term “combined preparation”, as used herein, refers to a preparation of multiple components. In some embodiments, the multiple components are thoroughly mixed at a molecular level. In other embodiments, the multiple components are maintained in separate volumes within a single product.

The term “omega-3 fatty acid”, as used herein, refers to a n-3 polyunsaturated fatty acid characterised by the presence of a double bond three atoms away from the terminal methyl group.

The term “positive modulator”, as used herein, refers to a substance which indirectly increases the effects of a primary ligand on a target protein.

The term “positive allosteric modulator”, as used herein, refers to a substance which indirectly induces an increase to the effects of an agonist on a target protein without directly activating the protein, by binding to a site distinct from the orthosteric binding site.

The term “a pharmaceutically acceptable salt thereof”, as used herein, means a salt formed by allowing the free form compound to react with an acid or base. Examples of pharmaceutically acceptable salts include hydrohalogenic acid salts such as hydrofluorides, hydrochlorides, hydrobromides, and hydroiodides; inorganic acid salts such as hydrochlorides, nitrates, perchlorates, sulfates and phosphates; lower alkanesulfonic acid salts such as methanesulfonates, trifluoromethanesulfonates, and ethanesulfonates; arylsulfonic acid salts such as benzenesulfonates, and p-toluenesulfonates; organic acid salts such as acetates, malates, fumarates, succinates, citrates, ascorbates, tartrates, oxalates, and maleates; alkali metal salts such as sodium salts, potassium salts, and lithium salts; alkaline earth metal salts such as calcium salts and magnesium salts; metal salts such as aluminum salts and iron salts; inorganic salts such as ammonium salts; amine salts including organic salts such as t-octylamine salts, dibenzylamine salts, morpholine salts, glucosamine salts, phenylglycine alkyl ester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts, N-benzylphenethylamine salts, piperazine salts, tetramethylammonium salts, and tris(hydroxymethyl)aminomethane salts; and amino acid salts such as glycine salts, lysine salts, arginine salts, ornithine salts, glutamates, and aspartates.

The term “pharmaceutical composition”, as used herein, means a pharmaceutical preparation suitable for administration to an intended human or animal subject for therapeutic purposes.

The term “sequential administration”, as used herein, refers to administration of two products to a patient wherein the two products are not administered simultaneously. In some embodiments each instance of sequential administration means that the two products are each administered less than 5 days, 4 days, 3 days, 2 days or 1 day apart.

The term “treatment” as used herein refers to any partial or complete treatment and includes: inhibiting the disease or symptom, i.e. arresting its development; and relieving the disease or symptom, i.e. causing regression of the disease or symptom.

### BRIEF DESCRIPTION OF THE FIGURES

FIG. 1 shows DHA effect on degradation of Aβ40 in a THP-1 cell model. Each degraded Aβ peptide is a product of two cleavages. The x-axis shows after which amino acid the cleavage occurred, and the y-axis counts each time the respective cleavage is detected. The peptide list for one group is an accumulation of detected identities. Three parallels were analysed for each condition/sample group. DHA: Docosahexaenoic acid.

FIG. 2 shows the cut pattern for Aβ in ex-vivo monocytes from (black columns) as well as THP-1 cells (grey columns). Each Aβ peptide is a product of two cleavages. The x-axis shows after which amino acid the cleavage occurred, and the y-axis counts each time the respective cleavage is detected. The peptide list for one group is an accumulation of detected identities. “Monocytes from donors (n=12)” refers both to monocytes from healthy donors and donors with Alzheimer's disease;

FIG. 3 shows a comparison of monocytic processing of Aβ40. All cut sites for the detected Aβ derived peptides were assessed, counted in regards to each of their peptide bond and summed up for the seven experiments (n=7 DHA stimulated+7 controls). The x-axis annotates the peptide bond number and the y-axis annotates the number of times each peptide bond is broken.

FIG. 4 shows monocytic expression of CHRNA7 in TPA differentiated THP-1 cells (control), and in TPA differentiated THP-1 cells with added Aβ42 peptides, Aβ42 peptides in combination with DHA and DHA alone. The y-axis shows the 56 kDa band signal intensity, stained with a CHRNA7-specific antibody (cat no 21379-1-AP, Proteintech) whereas the x-axis shows the different experimental conditions. DHA: Docosahexaenoic acid, Aβ42 peptides: the conventional amyloid beta peptide containing 42 amino acids. TPA: the phorbol ester 12-O-tetradecanoyl phorbol-13-acetate.

FIG. 5 shows monocytic expression (Western blot) of CHRNA7 and CHRFAM7A in differentiated THP-1 cells with added Aβ peptides, Aβ peptides in combination with DHA and DHA alone. DHA: Docosahexaenoic acid, Aβ1-40 peptides: the conventional amyloid beta peptide containing 40 amino acids.

FIG. 6 shows monocytic expression (quantitative PCR data) of CHRNA7 and CHRFAM7A with added Aβ peptides, Aβ peptides in combination with DHA and DHA alone.

FIG. 7 shows quantitative PCR measures of CHRNA7 (“N”; light grey) CHRFAM7A (“M”; black) and ratios (“N/M”; dark grey) in THP-1 monocyte cultures under different stimulatory condition (1-9), all values relative to TPA-treated but otherwise unstimulated condition (=1 at the y-axis). DHA: Docosahexaenoic acid, Gal: Galantamine, a PAM type 1, PNU: PNU-120596, a PAM type 2.

### DETAILED DESCRIPTION

The invention relates, in general terms, to a combination of an FFAR4 agonist and an α7 nAChR agonist or positive modulator, for the treatment of neurodegenerative diseases. The FFAR4 agonist and the α7 nAChR agonist or positive modulator may be administered as separate compositions, or they may be in the same composition.

FFAR4 Agonists

In some embodiments, FFAR4 agonist is one of a PUFA (polyunsaturated fatty acid), Compound A, NCG 21, GW9508 and TUG-891, or a pharmaceutically acceptable salt thereof. In some embodiments, the PUFA is α-linolenic acid (ALA), eicosapentaenoic acid (EPA) or docosahexaenoic acid (DHA). Preferably, the PUFA is an omega-3 fatty acid, more preferably DHA.

In some embodiments, more than one FFAR4 agonist is administered, selected from one or more PUFAs, GW9508 and TUG-891, or a pharmaceutically acceptable salt thereof. The one or more PUFA may be one or more of ALA, EPA and DHA. For example, the FFAR4 agonist may comprise two or more PUFAs, and may, optionally, further comprise one or both of GW9508 and TUG-891, or a pharmaceutically acceptable salt thereof. In another example, the FFAR4 agonist may be one PUFA and one or both of GW9508 or TUG-891, or a pharmaceutically acceptable salt thereof. In a further example, the FFAR4 agonist may be both GW9508 and TUG-891, or a pharmaceutically acceptable salt thereof. When there are two or more PUFAs, any combination of ALA, EPA and DHA may be used.

In some embodiments, the FFAR4 agonist may comprise EPA and DHA. In these embodiments, various ratios of EPA:DHA may be selected. In some embodiments, the FFA4 agonist is DPA (22:5), EPA (20:5) or ARA (20:4) or combinations of several PUFAs (such as in capsules).

The FFAR4 agonists may be naturally-occurring agonists, such as those found in natural oil, or may be synthetic agonists. For example, the FFAR4 agonists may be found naturally, for example, in fish oil, such as from herring or sardines, or the FFAR4 agonists may have been synthesised.

In some embodiments, the FFAR4 agonist is selected from the following: capric acid (10:0), undecyclic acid (11:0), lauric acid (12:0), tridecylic acid (13:0), myristic acid (14:0), pentadecanoic acid (15:0), palmitic acid (16:0), myristoleic acid (14:1n-5), palmitoleic acid (16:1n-7), oleic acid (18:1n-9), petroselinic acid (18:1n-12), cis-vaccenic acid (18:1n-7), elaidic acid (trans-18:1n-9), vaccenic acid (trans-18:1n-7), eicosenoic acid (20:1n-9), erucic acid (22:1n-9), nervonic acid (24:1n-9), linoleic acid (18:2n-6), γ-linoleic acid (18:3n-6), linolelaidic acid (all-trans-18: 2n-6), eicosadienoic acid (20:2n-6), dihomo-γ-linoleic acid (20:3n-6), arachidonic acid (20:4n-6), adrenic acid (22:4n-6), pinoleic acid (5,9,12-18:3n-6), α-linolenic acid (18:3n-3), stearidonic acid (18:4n-3), eicosatrienoic acid (20:3n-3), EPA (20:5n-3), docosatrienoic acid (22:3n-3), DHA (22:6n-3), c9,t11-conjugated linoleic acid (CLA) (c9,t11-18:2n-7), t9,t11-CLA (t9,t11-18:2n-7), t10,c12-CLA (t10,c12-18:2n-6), α-eleostearic acid (c9,t11,t13-18:3n-5), ximenynic acid, α-linolenic acid, Metabolex compound B, Metabolex 36, Merck cpdA, Banyu cpd 2, GSK137647A, TUG-1197, docosahexaenoic acid (22:6; DHA, w3), eicosapentaenoic acid (20:5; EPA, w3), stearic acid (18:0), cis-11,14,17-eicosatrienoic acid (20:3), cis-5,8,11,14,17-eicosapentaenoic acid (20:5; EPA), AMG-837, AMG-1638, ANT203, AS2034178, DC260126, glucagon-like peptide 1, GW1100, NCG21, TAK-875 (fasiglifam), TUG-469, TUG-424 or TUG-770.

In some embodiments, the FFAR4 agonist and, in particular, the PUFA described above is in the form of a free fatty acid. In other embodiments, it is provided in a different or derivative form and is, for example an ether (e.g. ethyl ether), ester or mono-, di-, or triglyceride thereof.

In some embodiments, the FFAR4 agonist is formulated with surfactants in order to provide a self-microemulsifying drug delivery system (SMEDDS). WO2010/119319 (which is incorporated herein by reference) discloses compositions of PUFAs, such as EPA and DHA, formulated with surfactants. Such formulation can improve the release and enhance solubilisation, digestion, bioavailability and/or absorption of the PUFA.

α 7 nAChR Agonist or Positive Modulator

In some embodiments, the α7 nAChR agonist or positive modulator is an agonist. In some embodiments, the α7 nAChR agonist is PNU-282907, SEN 12333, TC 5619, S24795 or A-582941, or a pharmaceutically acceptable salt thereof. In other embodiments, the α7 nAChR agonist is selected from the following list: GTS-21/DMXB-A, AR-R17779, SSR180711, ABBF, EVP-6124, TC-5619, RG3487, PHA-568487, AZD0328, ABT-107, and JN403.

In some embodiments, the α7 nAChR agonist or positive modulator is a positive modulator. In some embodiments, the positive modulator is a positive allosteric modulator. In some embodiments, the α7 nAChR positive modulator is Galantamine, NS-1738, PNU-120596 or TQS (RnDsystems. Cat no 4233/10), or a pharmaceutically acceptable salt thereof.

In some embodiments, the positive modulator is a Type I PAM. In some particular embodiments, the Type I PAM is selected from the following: Genistein, NS-1738, AVL-3288 and Galantamine. In some embodiments, the positive modulator is a Type II PAM. In some particular embodiments, the Type II PAM is selected from the following: PNU-120596 and PAM-2.

In further embodiments, the α7 nAChR agonist or positive modulator is selected from the following: Encenicline (EVP-6164), AQ051, ABT-126, Tropisetron, TC-5619, JNJ-39393406, nicotine and opipramol, AVL-8168, BMS-910731, BNC-210, BNC-375, bradanicline, EPGN-1137, Gln-1062, NBP-14, SKL-20540 and VQW-765.

Further details of suitable α7 nAChR agonists or positive modulators are provided in Jeremias Corradi and Cecilia Bouzat. Mol Pharmacol 90:288-299, September 2016 (in particular Table 1 thereof); Antonella De Jaco, Laura Bernardini, Jessica Rosati and Ada Maria Tata. Central Nervous System Agents in Medicinal Chemistry, 2017, 17 (in particular Table 1 thereof); Jason R. Tregellas, Korey P. Wylie Nicotine & Tobacco Research, 2018, 1-8 (in particular Table 1 thereof); and Neuronal Acetylcholine Receptor Subunit Alpha 7 (CHRNA7)—Pipeline Review, H2 2018, each of which is incorporated herein by reference.

In some embodiments, there is more than one α7 nAChR agonist and/or positive modulator. For example, there may be more than one of PNU-282987, Galantamine, NS-1738, PNU-120596 or TQS, or a pharmaceutically acceptable salt thereof, in any combination. In some embodiments, the α7 nAChR agonist or positive modulator comprises Galantamine, NS-1738, PNU-120596 and TQS, or a pharmaceutically acceptable salt thereof. In other embodiments, the α7 nAChR agonist or positive modulator consists of Galantamine, NS-1738, PNU-120596 and TQS.

Pharmaceutical compositions comprising the FFAR4 agonist and/or the α7 nAChR agonist or positive modulator are also provided herein. The pharmaceutical composition may further comprise at least one pharmaceutically acceptable carrier, diluent and/or excipient. In some embodiments, the pharmaceutical composition further comprises one or more additional active ingredients and/or adjuvants. In certain embodiments the pharmaceutical composition may further comprise one or more ingredients therapeutically effective for the same disease indication.

Specific Combinations

In some embodiments, the FFAR4 agonist is a PUFA, and the α7 nAChR agonist or positive modulator is an allosteric positive modulator. In some embodiments, the FFAR4 agonist is DHA and the α7 nAChR agonist or positive modulator is one or more of Galantamine, NS-1738, PNU-1205976 and TQS. In some embodiments, the FFAR4 agonist is DHA and the α7 nAChR agonist or positive modulator is Galantamine, NS-1738, PNU-1205976 and TQS.

Kits

In some embodiments, the FFAR4 agonist and the α7 nAChR agonist or positive modulator are provided as a single composition. In some embodiments, the FFAR4 agonist and the α7 nAChR agonist or positive modulator are provided as a kit comprising a first product which comprises the FFAR4 agonist and a second product which comprises the α7 nAChR agonist or positive modulator. The products may be administered separately to the patient, or may be formulated into a single composition which is then administered to the patient.

In some embodiments, the products are pharmaceutical products. In other embodiments, the kit further provides at least one pharmaceutically acceptable carrier, diluent and/or excipient for making up the FFAR4 agonist and/or α7 nAChR agonist or positive modulator into a pharmaceutical composition.

In embodiments, where there is more than one FFAR4 agonist and/or more than one α7 nAChR agonist and/or positive modulator, each FFAR4 agonist and/or each α7 nAChR agonist and/or positive modulator may be provided in a separate product. In some embodiments, all FFAR4 agonists are provided in a first product, and all α7 nAChR agonists and/or positive modulators are provided in second product.

Each product in the kit is provided in separate vials or compartments. The kit may further comprise instructions for administration of each product.

Neurodegenerative Diseases

The compositions of the present invention are for the treatment of neurodegenerative diseases, preferably in humans. In some embodiments, the neurodegenerative disease is associated with inflammation and a decrease in the expression of, or responsivity of, α7 nAChR. In some embodiments, the neurodegenerative disease is Alzheimer's disease.

Method of Treatment

There is also provided a method of treatment of neurodegenerative diseases, particularly in humans. In some embodiments, the method comprises administering, to a patient in need thereof, an FFAR4 agonist and an α7 nAChR agonist or positive modulator, as described above. The FFAR4 agonist and the α7 nAChR agonist or positive modulator may be administered as a single composition or may be administered as separate compositions.

In some embodiments, the FFAR4 agonist and the α7 nAChR agonist or positive modulator are administered simultaneously as separate compositions. In some embodiments, this simultaneous administration means that the two compositions are administered within a few minutes of each other (i.e. they are not administered at exactly the same time).

In some embodiments, the FFAR4 agonist and the α7 nAChR agonist or positive modulator are administered sequentially, i.e. one after the other. In some embodiments, the FFAR4 agonist is administered before the α7 nAChR agonist or positive modulator. In some embodiments, the α7 nAChR agonist or positive modulator is administered before the FFAR4 agonist. In some embodiments, the FFAR4 agonist is administered at least one week, at least two weeks, at least three weeks, at least one month, at least two months or at least three months before the α7 nAChR agonist or positive modulator. In some embodiments, the FFAR4 agonist is administered one week, two weeks, three weeks, one month, two months or three months before the α7 nAChR agonist or positive modulator. In some embodiments, the FFAR4 agonist is administered one month before the α7 nAChR agonist or positive modulator. The delay between the administrations does not have to be exact (i.e. exactly one week or exactly one month). Where the delay is in terms of weeks, a “week” is understood to mean 6 to 8 days. Where the delay is in terms of months, a “month” is understood to mean 28 to 32 days.

In some embodiments, the FFAR4 and α7 nAChR agonist or positive modulator are each administered several times (i.e. more than once) to the patient. In some embodiments, the FFAR4 agonist and the α7 nAChR agonist or positive modulator are administered the same number of times. In some embodiments, the FFAR4 agonist is administered a greater number of times than the α7 nAChR agonist or positive modulator. In some embodiments, the α7 nAChR agonist or positive modulator is administered a greater number of times than the FFAR4 agonist.

Each of the FFAR4 agonist and the α7 nAChR agonist or positive modulator may be, independently, administered at least twice, at least three times, at least four times, at least 5 times, at least 6 times, at least 7 times, at least 8 times, at least 9 times or at least 10 times. In some embodiments, each of the FFAR4 agonist and the α7 nAChR agonist or positive modulator is administered more than 10 times to the patient.

In some embodiments, the FFAR4 is administered every one, two or three weeks, or every one, two or three months. In some embodiments, the α7 nAChR agonist or positive modulator is administered every one, two or three weeks or every one, two or three months. When the FFAR4 agonist and the α7 nAChR agonist or positive modulator are in a single composition, the composition may be administered every one, two or three weeks or at least every one, two or three months.

In some embodiments, the method of treatment comprises diagnosing whether a subject has a neurodegenerative disease and, if so, administering the FFAR4 agonist and the α7 nAChR agonist or positive modulator, either as separate compositions, or as a single composition.

Dosages

In some embodiments, the DHA or derivative thereof is administered in an amount of at least 0.75 g per day 0.8 g per day, 0.85 g per day, 0.9 g per day, 1.0 g per day, 1.05 g per day, 1.1 g per day, 1.15 g per day, 1.2 g per day, 1.25 g per day, 1.3 g per day, 1.35 g per day, 1.4 g per day, 1.45 g per day and 1.5 g per day. In some embodiments, the DHA or derivative thereof is administered in an amount of no more than 4.5 g per day, 4.0 g per day, 3.95 g per day, 3.9 g per day, 3.85 g per day, 3.8 g per day, 3.75 g per day, 3.7 g per day, 3.65 g per day, 3.6 g per day, 3.55 g per day, 3.5 g per day, 3.45 g per day, 3.4 g per day, 3.35 g per day, 3.3 g per day, 3.25 g per day, 3.2 g per day, 3.15 g per day, 3.1 g per day, 3.0 g per day, 2.95 g per day, 2.9 g per day, 2.85 g per day, 2.8 g per day, 2.75 g per day, 2.7 g per day, 2.65 g per day, 2.6 g per day, 2.55 g per day, 2.5 g per day, 2.45 g per day, 2.4 g per day, 2.35 g per day, 2.3 g per day, 2.25 g per day, 2.2 g per day, 2.15 g per day, 2.1 g per day, 2.05 g per day, 2.0 g per day, 1.95 g per day, 1.9 g per day, 1.85 g per day, 1.8 g per day, 1.75 g per day, 1.7 g per day, 1.65 g per day, 1.6 g per day, 1.55 g per day or 1.5 g per day. In some embodiments the DHA or derivative thereof is administered in an amount between 0.75 g per day and 2.5 g per day, between 0.75 g per day and 2.25 g per day, between 0.8 g per day and 2.25 g per day, between 1.0 g per day and 2.0 g per day, between 1.25 g per day and 2.0 g per day, between 1.35 g per day and 2.0 g per day or between 1.5 g per day and 2.0 g per day. In some embodiments, the DHA or derivative thereof is administered in an amount of 1.5 g per day. In some embodiments, the DHA or derivative thereof is administered in an amount of 2.0 g per day. For FFAR4 agonists other than DHA or a derivative thereof, the dosage selected is one which achieves an equivalent effect to the dosages of DHA listed above. In embodiments where there is more than one FFAR4 agonist, the amount of each FFAR4 agonist administered may be, independently, as described above. In some embodiments, the total amount of FFAR4 agonist administered is as described above. For example, in some embodiments, the total amount of DHA or derivative thereof administered is 1.5 g per day. In other embodiments, the total amount of DHA or derivative thereof administered is 2.0 g per day. In other embodiments, the total amount of DHA or derivative thereof administered is between 3.5 g and 4.5 g per day, preferably 4.0 g per day. It is particularly preferred that the concentration of DHA or a derivative thereof administered is between 1 and 100 μM, preferably between 5 and 20 μM, more preferably between 8 and 12 μM, more preferably 10 μM. In some embodiments, the FFAR4 agonist is provided as a PUFA composition comprising at least 60% by weight of one or more PUFAs, such as at least 70%, 80%, 90% or 95% by weight of one or more PUFAs. In some embodiments, the FFAR4 agonist comprises at least 90% by weight of DHA.

In some embodiments, the α7 nAChR agonist or positive modulator is administered in an amount of at least 4 mg per day, at least 5 mg per day, at least 6 mg per day, at least 7 mg per day, at least 8 mg per day, at least 9 mg per day, at least 10 mg per day, at least 11 mg per day, at least 12 mg per day, at least 13 mg per day, at least 14 mg per day, at least 16 mg per day, at least 17 mg per day, at least 18 mg per day, at least 19 mg per day, at least 20 mg per day, at least 21 mg per day, at least 22 mg per day, at least 23 mg per day or at least 24 mg per day. In some embodiments, the α7 nAChR agonist or positive modulator is administered in an amount of no more than 30 mg per day, no more than 29 mg per day, no more than 28 mg per day, no more than 27 mg per day, no more than 26 mg per day, no more than 25 mg per day or no more than 24 mg per day. In some embodiments, the α7 nAChR agonist or positive modulator is administered in an amount between 4 mg per day and 24 mg per day, between 5 mg per day and 24 mg per day, between 5 mg per day and 10 mg per day, between 8 mg per day and 24 mg per day, between 8 mg per day and 16 mg per day, or between 16 mg per day and 24 mg per day. Further details of suitable dosage may be found in Wattmo et al. Alzheimer's Research & Therapy 20135:2, which is incorporated herein by reference. In embodiments where there is more than one α7 nAChR agonist and/or positive modulator, each agonist and/or positive modulator is, independently, administered in an amount as described above. In some embodiments, the total amount of the one or more α7 nAChR agonist or positive modulator administered is as described above.

Administration

The FFAR4 agonist and α7 nAChR agonist or positive modulator may be administered to a patient by any delivery technique known to those skilled in the art. For example, among other techniques, the FFAR4 agonist and α7 nAChR agonist or positive modulator may be administered to a subject by injection, orally, in the form of a solution, in the form of liposomes or in dry form (for example, in the form of coated particles, capsules for oral intake, etc) or by means of a dermatological patch. In embodiments where the FFAR4 agonist and the α7 nAChR agonist or positive modulator are administered as separate compositions, they may be administered by the same or different techniques. In some embodiments, the FFAR4 agonist is administered orally. In some embodiments, the α7 nAChR agonist or positive modulator is administered orally.

### EXAMPLES

### Example 1

Results from the following pilot experiments demonstrate that an Immunoprecipitation Liquid Chromatography Mass Spectrometry (IP LC-MS) approach detects Abeta degradation relevant for monitoring of both disease progression and treatment. The IP LC-MS tool has been used for two sets of samples; a cell model system and on biological fluid from patients and healthy subjects.

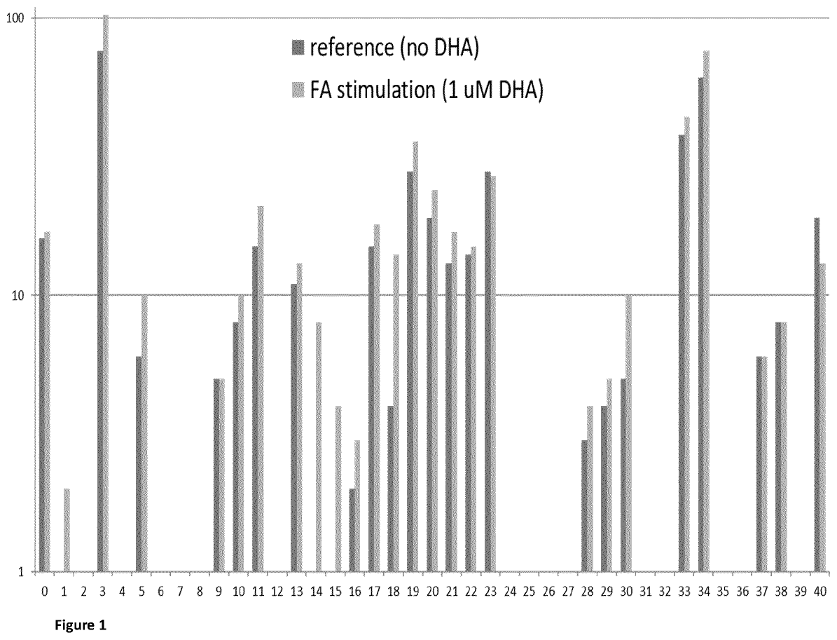

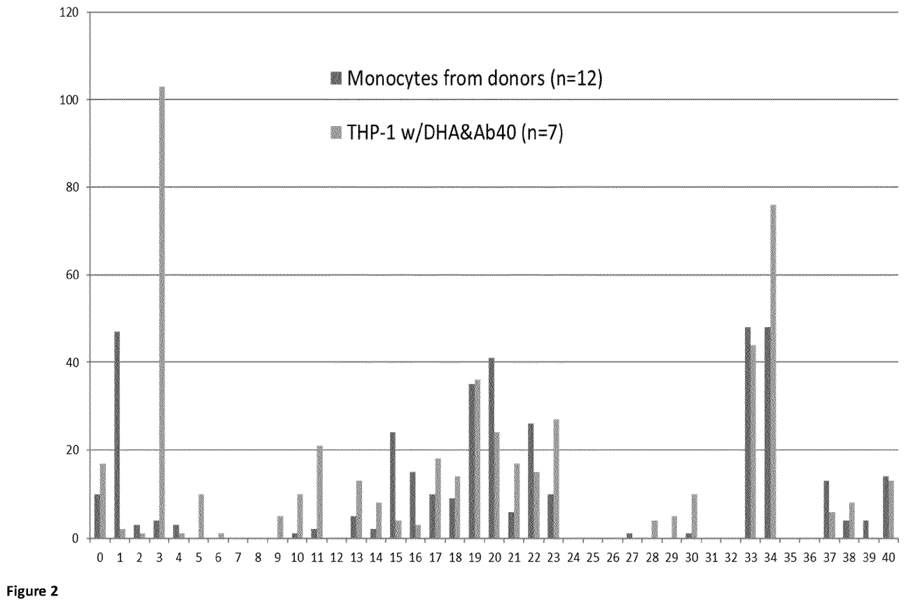

Firstly, a cell model was used to study the effect of the omega-3 fatty acid DHA on degradation of amyloid beta. Here, the THP-1 cells were incubated with and without DHA (1 μM), and subsequently with Abeta (1-40 aa, 10 ng/μL). Secondly, monocyte from healthy controls (NC) and patients with neurodegenerative diseases (AD) were isolated. In both these cases, the cells were lysed and IP LC-MS was performed. The peptide identified from IP LC-MS gave rise to the illustration of Abeta cut patterns shown in FIG. 1 and FIG. 2.

In FIG. 1, each bar in the graph represents the accumulated cleavage sites on each position along the 40 amino acids in Abeta 1-40. Thus the bar contains peptides of various lengths, but with the same start or end amino acid. Three parallels were analysed for each condition/sample group, which refers to the triplicate incubations of each condition, with or without DHA.

The cut pattern from the DHA experiment (FIG. 1) implies differing enzymatic activities between cells that are subjected and not subjected to DHA. Similarly, the cut pattern obtained for Abeta derived from cells from healthy and diseased subjects are different and in part comparable to those from the THP-1 model. FIG. 2 illustrates that the cut sites in the THP-1 cells correspond to the cut sites in the donor monocytes. It is envisaged that further experiments will screen various compounds for effects on Abeta degradation, and other disease-related protein entities.

### Example 2

Monocytic THP-1 cells were used as a model system, and IP LC-MS as analytical approach to investigate the effect of DHA on monocytic Abeta-40 processing.

A THP-1 cell line culture was matured and differentiated, split to be control and stimulated parallels and this was replicated to be performed a total of 7 times (controls n=7; DHA stimulated n=7). Test cells were incubated with DHA overnight, and all samples were incubated with Abeta-40 for 1 to 2 hours. The cells were lysed by freeze-thaw cycles prior to immunoprecipitation performed with two commercial and one in-house antibody. The immunoprecipitate was injected into an LC-MS system. The liquid chromatography was operated in a conventional two column setup with C4 sorbent. The mass spectrometry was operated in conventional ESI+ and DDA mode.

In the cell lysate, intact Abeta-40 was sparsely detected, whilst Abeta-40 degradation products were widely detected proving both monocytic engulfed and degraded Abeta-40. An accumulated number of 89 degraded Abeta peptides were identified in the samples analysed (n=14).

The Abeta-40 peptides between the conditions were also semi-quantitatively evaluated. Here, the catabolic peptide yield was compared, with an average ratio of 1.3 (12% RSD) of catabolic peptides in DHA versus control samples. This implies that DHA functions as a catalyst for either or both monocytic phagocytosis and catabolism of Abeta-40.

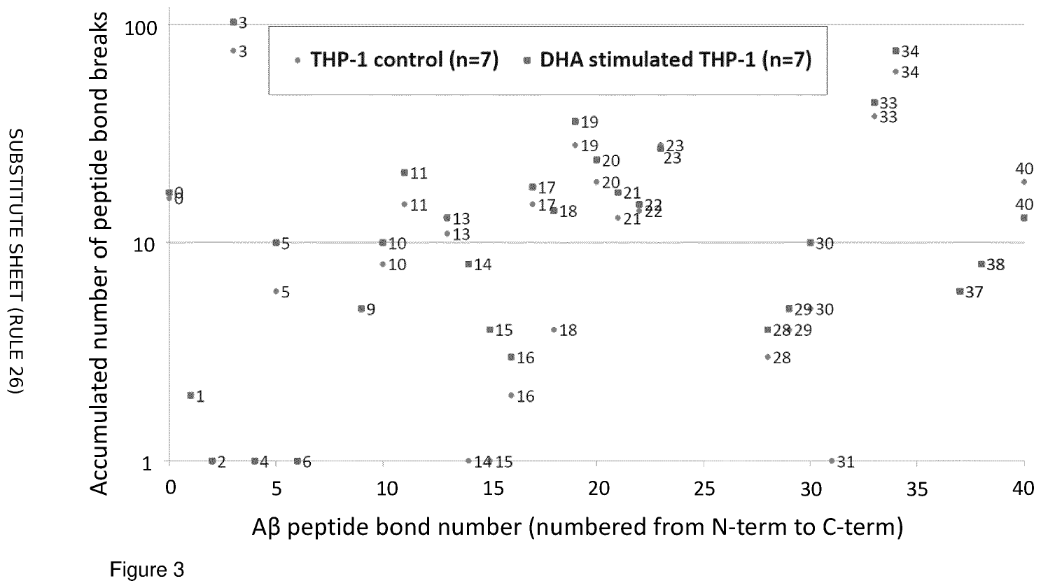

The Abeta cell culture degradation patterns are shown in FIG. 3. The results harmonize in vitro experiments for lysosomal degradation and that obtained from patient harvested monocytes, which indicates that comparable effects are plausible in vivo.

### Example 3

Ex Vivo Monocytes

Monocytes were isolated from donor blood samples (n=36) with an age range from 24 to 84 years and gender distribution of 1:1. IP and nLCMS were performed to investigate the monocytic Aβ products. The cells were lysed by freeze-thaw cycles prior to immunoprecipitation (IP) performed with two commercial and one in-house antibody.

The IP eluate was injected to an nLC-MS system. The nLC was operated in a conventional two column setup with C4 sorbent. The MS was operated in conventional ESI+ and DDA mode.

An accumulated number of 38 endogenous Aβ peptides was identified in monocytes. These peptides predominantly derive from the following pbbs; 13-23, 33-34 and 37-40, as shown in FIG. 2 demonstrating a conserved segment around the mid-domain similar to results from the endolysosomal model(1).

THP-1 Cells

Monocytic THP-1 cells were used as a model system and IP and nLCMS as analytical approaches to investigate DHA's effects on monocytic Aβ 1-40 processing: A THP-1 cell line culture was matured and differentiated, split to be control (7) and stimulated parallels (7). The stimulated samples were incubated with DHA overnight, and all samples were incubated with Aβ 1-40 for 1 or 2 h. IP and nLCMS was performed as above (FIGS. 1, 2 and 3).

Western Blot Protein Analysis

The cellular samples were tested for the presence of the α7 subtype of the nicotinic acetylcholine receptor (nAChR) by Western blot analysis on THP-1 cells that were incubated with and without Abeta1-42 and with and without DHA. The purpose of this was to show monocytic membrane expression of nAChR and to explore altered regulation of this receptor in response to DHA stimulation.

THP-1 Cell Growth

The THP-1 cells were seeded in 6-well plates with 2 mL per well at a concentration of 830 000 cells/mL (experiment 1) or 860 000 cells/ml (experiment 2), and differentiated using 100 nM TPA (12-O-Tetradecanoylphorbol-13-Acetate) for 24 hours. For the experiments, DHA was added to give a concentration of 100 uM (experiment 1) or 10 uM and 100 uM (experiment 2), and Aβ42 was added at a final concentration of 2.5 ng/ul.

The cells were incubated overnight (18 hours). Each DHA experiment had parallels of cells not incubated with DHA. After incubation the cells were kept cold, scraped loose and transferred to 15-ml tubes. Cells were washed twice with cold PBS before resuspended in 100 ul PBS and transferred to an Eppendorf tube. The cells were lysed through five freeze thaw cycles, and total protein in each sample was determined by the BCA protein assay. Samples were stored at −80° C. upon analysis.

Western Blot Conditions

Western blot analysis was performed cat no 21379-1-AP, Proteintech, using 1:1000 using dilution. The secondary antibody was a goat anti-rabbit IgG-HRP (cat no 4030-05, Southern Biotech) diluted 1:2000. Solvents for dilutions were as described below.

Samples were dissolved in 4× Laemmli buffer w/ b-ME (BioRad and, respectively) denatured at 95° C. for 5 min, and a quantity of 12 μg protein/sample/well was loaded to the gel. A volume of 10 uL of Precision Plus protein Dual Xtra Color Standards (BioRad) was used for molecular weight estimation. The samples were resolved in 8-16% gradient SDS-PAGE (Criterion TGX precast gels, BioRad) and immunoblotted onto PVDF membranes (GE Healthcare). Membranes were blocked in 5% non-fat dried milk in 1× Tris Buffered Saline containing 0.1% Tween20 (1×TBS-T) (BioRad) at room temperature for 1 h and incubated overnight at 4° C. with primary antibodies in 1×TBS-T with 1% non-fat dried milk. After washing, the membranes were incubated with secondary antibody in 5% non-fat dried milk in 1×TBS-T for 1 h at room temperature. The blots were visualized by ECL Plus Western blotting detection system (GE Healthcare) according to the supplier's instructions. Membranes were visualized on the LAS-3000 mini (Fujifilm Corporation) and band intensities were quantified using MultiGauge analysis software (Fujifilm Corporation).

Bands presented are at the predicted MW of the nAChR (56 kDa).

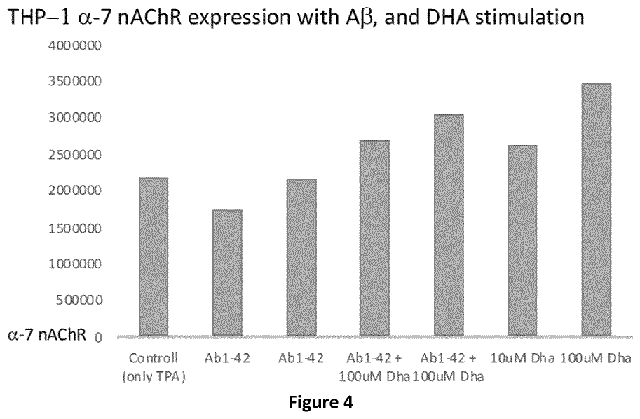

The results are shown in Table 1 and FIG. 4 (CHRNA7 is the 56 kDa protein).

TABLE 1
~56 kDa

| Exp | Lane | Sample | Band signal intensity |
|---|---|---|---|
| Exp | 1 | Control (only TPA) | 2.2E+06 |
| 1 | 2 | Ab1-42 | 1.7E+06 |
| | 3 | 100 uM Dha | 3.5E+06 |
| | 4 | Ab1-42 + 100 uM | 3.0E+06 |
| Exp | 5 | Ab1-42 | 2.1E+06 |
| 2 | 6 | Ab1-42 + 10 uM Dha | 2.6E+06 |
| | 7 | Ab1-42 + 100 uM | 2.7E+06 |
| | | Dha | |

### CONCLUSION

The results of the experiments are consistent with respect to CHRNA7 upregulation upon DHA stimulation (CHRNA7 is the 56 kDa protein), which thus accompany increased Ab degradation. There may also be a trend for lower expression with Ab42 only, possibly impeding Ab uptake.

### Example 5

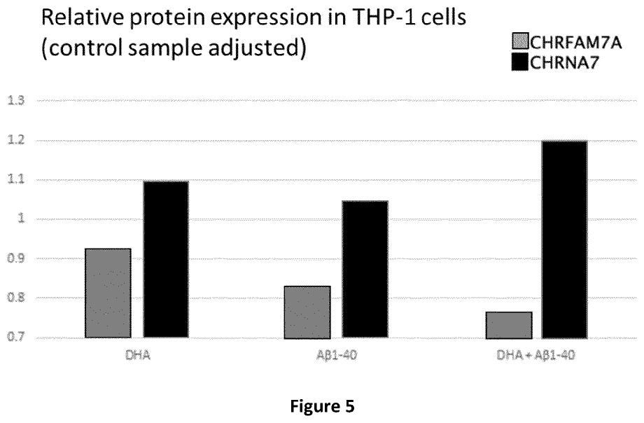

FIG. 5 shows monocytic expression (Western blot) of CHRNA7 and CHRFAM7A in differentiated THP-1 cells with added Aβ peptides, Aβ peptides in combination with DHA and DHA alone. DHA: Docosahexaenoic acid, Aβ1-40 peptides: the conventional amyloid beta peptide containing 40 amino acids.

The results from FIG. 5 show an increase in CHRNA7 (functional subunit) expression and a decrease in CHRFAM7A (subunit known to hinder α7 nAChR function) expression when stimulated with DHA. The effect is more pronounced with co-stimulation with DHA and Aβ1-40 peptide.

### Example 6

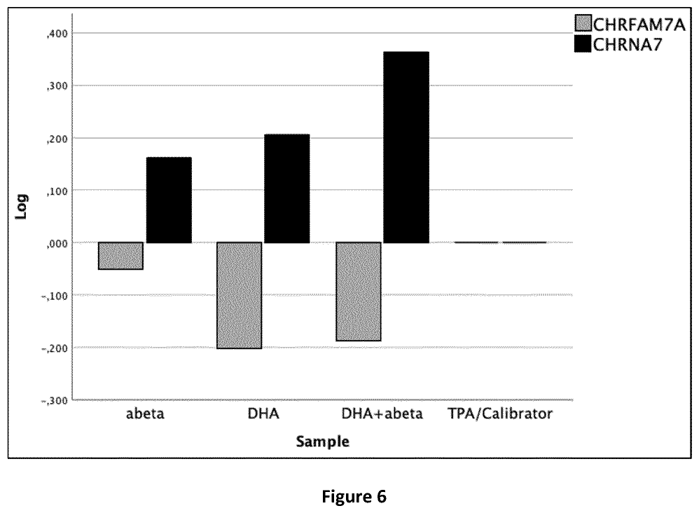

FIG. 6 shows monocytic expression (quantitative PCR data) of CHRNA7 and CHRFAM7A with added Aβ peptides, Aβ peptides in combination with DHA and DHA alone.

The results from FIG. 6 show an increase in CHRNA7 (functional subunit) transcription and a decrease in CHRFAM7A (subunit known to hinder α7 nAChR function) transcription when stimulated with DHA. The effect is more pronounced with co-stimulation with DHA and Aβ1-40 peptide.

### Example 7

THP-1 Cell Line and Treatments

The human acute monocytic leukemia cell line THP-1 (ATCC TIB-202, ATCC, US) was cultured in RPMI 1640 with GlutaMax (Gibco, Life Technologies, UK) supplemented with 10% fetal bovine serum (FBS), (Gibco, Life Technologies, UK) and 1% Antibiotic/Antimycotic (Gibco, Life Technologies, UK) at 37° C. and 5% CO2.

1,6 million cells were seeded in each well in 6-well plates and differentiated to macrophages by a 48 h treatment of 100 nM TPA (12-O-tetradecanoylphorbol-13-acetate), (Cell Signaling Technology, US). Cells were treated with 100 μM Docosahexaenoic acid (DHA), (Sigma Aldrich, Germany), 10 ng/ul Amyloid beta 1-40 (Aβ), (ApexBio, US), 10 μM PNU-120596, (Sigma Aldrich, Germany), 40 μM Galanthamine hydrobromide, (Sigma Aldrich, Germany) and combinations overnight (approximately 20 h).

RNA Isolation and Quantitative Real-Time PCR (qPCR)

Total RNA was isolated with the RNeasy Plus Mini kit (Qiagen) using genomic DNA eliminator columns. THP-1 cells were lysed directly in the wells using 350 μl RLT Plus according to the protocol and stored at −80° C. The frozen lysate was incubated at 37° C. in a water bath until completely thawed and homogenized using QIAshredder (Qiagen) spin columns. RNA was eluted in 30 μl RNase free water and the quantity assessed using the NanoDrop ND-1000 Spectrophotometer (NanoDrop Technologies).

qPCR Analysis

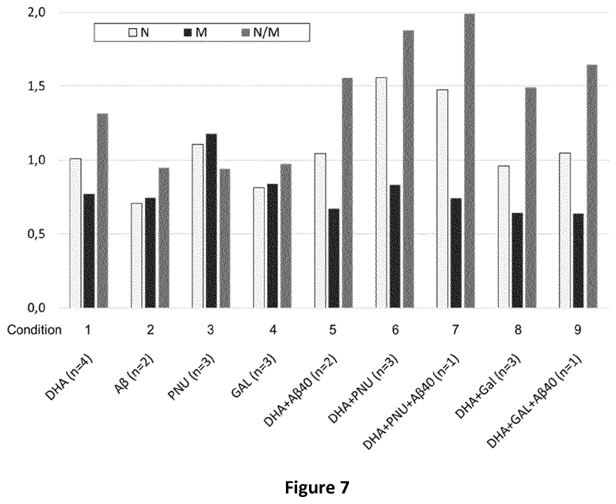

1 μg total RNA was reverse transcribed using the QuantiTect cDNA Reverse Transcription Kit (Qiagen). Due to low CHRNA7 expression (Cq values>38, Table 2), we increased RNA input to 2 μg, changed the reverse transcription kit to High Capacity Reverse Transcription Kit (Life Technologies AS) and the cDNA was preamplified using TaqMan PreAmp Master Mix (Life Technologies AS), running 18 cycles and diluted 1:20 (FIG. 7). We then obtained Cq values<27 (average 25,4) for CHRNA7 and <20 (average 18,6 for CHRFAM7A). Absolute quantification was run using CHRFAM7A and CHRNA7 synthetic oligonucleotides standards (GeneArt, Life Technologies AS). 2.5 ul cDNA diluted 1:20 after preamplification was applied per qPCR reaction using TaqMan gene expression assays CHRFAM7A (Hs04189909_m1) and CHRNA7 (Hs01063372_m1) (Thermo Fisher) and TaqMan Gene expression Master Mix (Life Technologies AS) in a total volume of 10 μl and run in triplicates on Quant Studio 7 (Applied Biosystems).

Discussion

Transcription of subclasses of α-7 nicotinic receptors (the recently discovered uniquely human CHRFAM7A (“M”) and the classic form CHRNA7 (“N”)) can be modified by combining DHA and α7-cholinergic activation in such a fashion that the N/M ratio is increased. CHRNA7 is the functional subunit whereas CHRFAM7A is a subunit known to hinder α7 nAChR function.

This Example presents evidence that innate immune α7-cholinergic (nicotinergic) responsiveness can be increased by DHA (Docosahexaenoic acid) and α7-allosteric positive modulators, as combined DHA and nicotinergic activation reduces CHRFAM7A-transcription and increases CH RNA7 transcription.

The results show that:

1) Transcription of subclasses of α7 nicotinic receptors (the recently discovered uniquely human CHRFAM7A and the classic form CHRNA7) can be modified by combining DHA and an α7-cholinergic allosteric modulator.

2) Receptor activation increases CHRNA7 and decreases CHRFAM7A transcription.

3) The results also support the model that CHRNA7 and CHRFAM7A transcription are independently regulated.

4) We show that CHRNA7 and CHRFAM7A subclasses are transcribed in monocytes.

FIG. 7 shows results from THP-1 monocytes grown in culture with TPA (12-O-tetra-decanoylphorbol-13-acetate) and different additional conditions. Quantitative PCR, demonstrating that CHRNA7 (“N”) transcription is stable whereas CHRFAM7 (“M”) transcription is reduced in condition 1 (DHA), leading to an increased N/M ratio/grey column). Condition 2, Amyloid 13, shows both reduced N and M receptor transcription. Condition 3 shows smaller changes in the presence of PNU-120596 (α-7 nicotinic positive modulator). Similarly, condition 4 shows smaller changes in the presence of GAL (Galantamine; α-7 nicotinic allosteric modulator). Condition 5, DHA+Amyloid β shows unaltered N and reduced M transcription, resulting in an increased N/M ratio. Condition 6 shows strongly increased N-receptor transcription in the presence of PNU and DHA. Condition 7 shows strongly increased N-receptor transcription in the presence of PNU and DHA and Amyloid β reduced M transcription and a strongly increased N/M ratio. Condition 8 shows reduced M-receptor transcription in the presence of GAL and DHA, and an increased N/M ratio. Condition 9 shows reduced M-receptor transcription in the presence of GAL and DHA and Amyloid 13, and an increased N/M ratio.

Receptor activation increases CHRNA7 transcription and decreases CHRFAM7 transcription.

Given the expected effects of CHRFAM7 expression on expression of functional α7 nicotinic receptors the observed high N/M ratios are expected to be beneficial and result from the proposed combined treatment regimens (FIG. 7).

With particular relevance for Alzheimer's disease, we show that DHA, with Amyloid β and with or without α7 positive modulators, increases N transcription and reduces M transcription, and thus skews the α7 subclass transcription towards a higher N/M ratio (FIG. 7).

We show that CHRNA7 and CHRFAM7A subclasses are transcribed in monocytes (Table 2).

TABLE 2

| | Cq | |
|---|---|---|
| Gene | values | Cell type |
| CHRNA7_Hs01063372_m1 | 39.7 | THP-1 monocytes |
| CHRFAM7A_Hs04189909_m1 | 32.4 | THP-1 monocytes |

### REFERENCES

1. Rogeberg M, Furlund C B, Moe M K, Fladby T. Identification of peptide products from enzymatic degradation of amyloid beta. Biochimie. 2014.
2. Olivera-Perez H M, Lam L, Dang J, Jiang W, Rodriguez F, Rigali E, et al. Omega-3 fatty acids increase the unfolded protein response and improve amyloid-beta phagocytosis by macrophages of patients with mild cognitive impairment. FASEB J. 2017; 31(10):4359-69.
3. Rothbard J B, Rothbard J J, Soares L, Fathman C G, Steinman L. Identification of a common immune regulatory pathway induced by small heat shock proteins, amyloid fibrils, and nicotine. Proc Natl Acad Sci USA. 2018; 115(27):7081-6.
4. Maldifassi M C, Martin-Sanchez C, Atienza G, Cedillo J L, Arnalich F, Bordas A, et al. Interaction of the alpha7-nicotinic subunit with its human-specific duplicated dupalpha7 isoform in mammalian cells: Relevance in human inflammatory responses. J Biol Chem. 2018; 293(36):13874-88.
5. Eduardo C C, Alejandra T G, Guadalupe D K J, Herminia V G, Lenin P, Enrique B V, et al. Modulation of the extraneuronal cholinergic system on main innate response leukocytes. J Neuroimmunol. 2019; 327:22-35.
6. De Lucas-Cerrillo et al. “Function of partially duplicated human alpha77 nicotinic receptor subunit CHRFAM7A gene: potential implications for the cholinergic anti-inflammatory response”. J Biol Chem. 2011; 286(1):594-606.
7. Maroli A, Di Lascio S, Drufuca L, et al. Effect of donepezil on the expression and responsiveness to LPS of CHRNA7 and CHRFAM7A in macrophages: A possible link to the cholinergic anti-inflammatory pathway. J Neuroimmunol 2019; 332:155-166.
8. Chan T, Williams E, Cohen O, Eliceiri B P, Baird A, Costantini T W. CHRFAM7A alters binding to the neuronal alpha-7 nicotinic acetylcholine receptor. Neurosci Lett 2019; 690:126-131.
9. Yang T, Xiao T, Sun Q, Wang K. The current agonists and positive allosteric modulators of alpha7 nAChR for CNS indications in clinical trials. Acta Pharm Sin B 2017; 7:611-622.
10. Lasala M, Corradi J, Bruzzone A, del Carmen Esandi M, Bouzat C. A human-specific, truncated α7 nicotonic receptor subunit assembles with full-length α7 and forms functional receptors with different stoichiometries. J Biol Chem 2018; 293(27): 10707-10717.
11. Nordengen K, Kirsebom B E, Henjum K, et al. Glial activation and inflammation along the Alzheimer's disease continuum. J Neuroinflammation 2019; 16:46.
12. Mongan D, Ramesar M, Focking M, Cannon M, Cotter D. Role of inflammation in the pathogenesis of schizophrenia: A review of the evidence, proposed mechanisms and implications for treatment. Early Intery Psychiatry 2019.
13. Stephenson J, Nutma E, van der Valk P, Amor S. Inflammation in CNS neurodegenerative diseases. Immunology 2018; 154:204-219.

### Further References

Alvarez-Curto et al. 2016. “Metabolism meets immunity: The role of free fatty acid receptors in the immune system”. Biochem. Pharmacol. 114, 3-13.
Antonietta Ajmone-Cat M, Lavinia Salvatori M, De Simone R, et al. “Docosahexaenoic acid modulates inflammatory and antineurogenic functions of activated microglial cells”. J Neurosci Res 2012; 90:575-587.
Araud et al. “The chimeric gene CHRFAM7A, a partial duplication of the CHRNA7 gene, is a dominant negative regulator of α7nAChR function”. Biochem Pharmacol. 2011 Oct. 15; 82(8): 904-914.
Benfante et al. “Expression of the alpha7 nAChR subunit duplicate form (CHRFAM7A) is down-regulated in the monocytic cell line THP-1 on treatment with LPS”. J Neuroimmunol. 2011; 230(1-2):74-84.
Chan et al. “CHRFAM7A alters binding to the neuronal alpha-7 nicotinic acetylcholine receptor”. Neurosci Lett. 2019; 690:126-31.
Costantini et al. “A Human-Specific alpha7-Nicotinic Acetylcholine Receptor Gene in Human Leukocytes: Identification, Regulation and the Consequences of CHRFAM7A Expression”. Mol Med. 2015; 21:323-36.
De Jaco A et al. “Alpha-7 nicotinic receptors in nervous system disorders: From function to therapeutic perspectives”. Cent Nery Syst Agents Med Chem, 2017, 17(2), 100-108.
De Lucas-Cerrillo et al. “Function of partially duplicated human alpha77 nicotinic receptor subunit CHRFAM7A gene: potential implications for the cholinergic anti-inflammatory response”. J Biol Chem. 2011; 286(1):594-606.
Freund Levi Y, Vedin I, Cederholm T, et al. “Transfer of omega-3 fatty acids across the blood-brain barrier after dietary supplementation with a docosahexaenoic acid-rich omega-3 fatty acid preparation in patients with Alzheimer's disease: the OmegAD study”. J Intern Med 2014; 275:428-436.
Im D S, (May 2015). “Functions of pomega-3 fatty acids and FFAR4 (GPR120) in macrophages”. Eur J Pharmacol S0014-299 (15), 00458-6.
King J R, Gillevet T C, Kabbani N. “A G protein-coupled alpha7 nicotinic receptor regulates signaling and TNF-alpha release in microglia”. FEBS Open Bio. 2017; 7(9):1350-61
Koronyo Y, Salumbides B C, Sheyn J, et al. “Therapeutic effects of glatiramer acetate and grafted CD115(+) monocytes in a mouse model of Alzheimer's disease”. Brain 2015; 138:2399-2422.
Krabbe G, Halle A, Matyash V, et al. “Functional impairment of microglia coincides with Beta-amyloid deposition in mice with Alzheimer-like pathology”. PLoS One 2013; 8:e60921.
Lappe J, Kunz I, Bendik I, Prudence K, Weber P, Recker R, Heaney R. “Effect of a combination of genistein, polyunsaturated fatty acids and vitamins D3 and K1 on bone mineral density in postmenopausal women: a randomized, placebo-controlled, double-blind pilot study”. Eur J Nutr 2013; 52:203-215.
Nordengen et al. “Glial activation and inflammation along the Alzheimer's disease continuum”. J Neuroinflammation. 2019; 16(1):46
Quik et al, “Alpha7 nicotinic receptors as therapeutic targets for Parkinson's disease”.

Biochem Pharmacol., 2015, 97(4), 399-407.
Sinkus et al. “The human CHRNA7 and CHRFAM7A genes: A review of the genetics, regulation, and function”. Neuropharmacology. 2015; 96(Pt B):274-88.
Tan Y, Ren H, Shi Z, et al. “Endogenous Docosahexaenoic Acid (DHA) Prevents Abeta1-42 Oligomer-Induced Neuronal Injury”. Mol Neurobiol 2016; 53:3146-3153.
Wang X, Hjorth E, Vedin I, et al. “Effects of n-3 FA supplementation on the release of proresolving lipid mediators by blood mononuclear cells: the OmegAD study”. J Lipid Res 2015; 56:674-681.
Zuroff L, Daley D, Black K L, Koronyo-Hamaoui M. “Clearance of cerebral Abeta in Alzheimer's disease: reassessing the role of microglia and monocytes”. Cell Mol Life Sci 2017; 74:2167-2201.

1-18. (canceled)

19. A combined preparation or composition comprising a free fatty acid receptor 4 (FFAR4) agonist and an α7 nicotinic acetylcholine receptor (nAChR) agonist or α7 nAChR positive modulator.

20. The combined preparation or composition according to claim 19, wherein the combined preparation or composition is a pharmaceutical composition and further comprises a pharmaceutically acceptable carrier, diluent or excipient.

21. The combined preparation or composition according to claim 19, wherein the FFAR4 agonist is one or more selected from a polyunsaturated fatty acid, Compound A, NCG 21, GW9508 and TUG-891, or a pharmaceutically acceptable salt of any thereof.

22. The combined preparation or composition according to claim 19, wherein the FFAR4 agonist is docosahexaenoic acid (DHA).

23. The combined preparation or composition according to claim 19, wherein the α7 nAChR agonist or α7 nAChR positive modulator is one or more selected from galantamine, NS-1738, PNU-120596 and TQS, or a pharmaceutically acceptable salt of any thereof.

24. The combined preparation or composition according to claim 19, wherein the α7 nAChR agonist or α7 nAChR positive modulator is one or more selected from PNU-282987, SEN 12333, TC 5619, S24795 and A-582941, or a pharmaceutically acceptable salt of any thereof.

25. The combined preparation or composition according to claim 19, wherein the α7 nAChR agonist or α7 nAChR positive modulator is a Type I positive allosteric modulator (PAM).

26. The combined preparation or composition according to claim 25, wherein the Type I PAM is selected from the group consisting of genistein, NS-1738, AVL-3288 and galantamine.

27. The combined preparation or composition according to claim 19, wherein the α7 nAChR agonist or α7 nAChR positive modulator is a Type II PAM.

28. The combined preparation or composition according to claim 27, wherein the Type II PAM is selected from the group consisting of PNU-120596 and PAM-2.

29. The combined preparation or composition according to claim 19, wherein the combined preparation or composition comprises more than one α7 nAChR positive modulator.

30. The combined preparation or composition according to claim 29, wherein the more than one α7 nAChR positive modulator comprises galantamine, NS-1738, PNU-120596 and TQS.

31. The combined preparation or composition according to claim 19, wherein the FFAR4 agonist is DHA and the α7 nAChR agonist or α7 nAChR positive modulator comprises galantamine, NS-1738, PNU-120596 and TQS.

32. The combined preparation or composition according to claim 19, wherein the FFAR4 agonist and α7 nAChR agonist or α7 nAChR positive modulator are maintained in separate volumes.

33. The combined preparation or composition according to claim 19, wherein the FFAR4 agonist and α7 nAChR agonist or α7 nAChR positive modulator are thoroughly mixed at a molecular level.

34. A method of treating a neurodegenerative disease, wherein the method comprises administering an FFAR4 agonist simultaneously or sequentially with an α7 nAChR agonist or α7 nAChR positive modulator to a patient in need thereof.

35. The method according to claim 34, wherein the neurodegenerative disease is Alzheimer's disease.

36. A method of treating a neurodegenerative disease, wherein the method comprises administering a combined preparation or composition according to claim 19 to a patient in need thereof.

37. The method according to claim 36, wherein the neurodegenerative disease is Alzheimer's disease.

38. A kit comprising a first product comprising an FFAR4 agonist and a second product comprising an α7 nAChR agonist or α7 nAChR positive modulator.
